(19) United States
(12) Patent Application Publication
(10) Pub. No.: US 2026/0047781 A1
(43) Pub. Date: Feb. 19, 2026

(54) POROUS MICRONEEDLE ARRAY-BASED INTERSTITIAL FLUID GLUCOSE DETECTION DEVICE, METHOD OF PREPARING POROUS MICRONEEDLE ARRAY WITH POROSITY GRADIENT, AND METHOD OF PREPARING COLORIMETRIC FILM

(71) Applicant: WUHAN UNIVERSITY, Hubei (CN)

(72) Inventors: Hui LI, Hubei (CN); Yunfan ZHANG, Hubei (CN); Shengnan SHEN, Hubei (CN); Hua TU, Hubei (CN)

(73) Assignee: WUHAN UNIVERSITY, Hubei (CN)

(21) Appl. No.: 18/924,939

(22) Filed: Oct. 23, 2024

(30) Foreign Application Priority Data
Aug. 16, 2024 (CN) 202411128158.7

(51) Int. Cl. A61B 5/145

(52) U.S. Cl. CPC A61B 5/14514; A61B 5/14532

(57) ABSTRACT

A porous microneedle array-based interstitial fluid glucose detection device includes a microneedle module, a colorimetric detection module, an electrochemical detection module, and a liquid transport layer. The microneedle module includes a porous microneedle array with a porosity gradient, the porosity gradually increases from the needle tip to the microneedle substrate; the porous microneedle array penetrates human epidermis and extract interstitial fluid, which is then transferred to both the colorimetric detection module and the electrochemical detection module via the liquid transport layer. Within the colorimetric detection module, glucose undergoes a reaction with the color on the colorimetric film, resulting in color development that enables rapid glucose detection; In the electrochemical detection module, glucose undergoes a redox reaction on the working electrode, resulting in the generation of a current signal for the accurate detection of glucose.

### CROSS-REFERENCE TO RELATED APPLICATION

This application claims the priority benefit of China application serial no. 202411128158.7, filed on Aug. 16, 2024. The entirety of the above-mentioned patent application is hereby incorporated by reference herein and made a part of this specification.

### TECHNICAL FIELD

The present application relates to the field of medical devices, specifically focusing on an interstitial fluid glucose detection device that is based on a porous microneedle array.

### BACKGROUND

The increasing of diabetic patients and early-stage groups highlights the significance of accurate detection of blood glucose levels for self-health management and timely treatment. While the traditional fingertip blood collection method is known for its accuracy, it is also cumbersome and accompanied by unavoidable pain. Therefore, there is a pressing need to develop a blood glucose testing technique that is accurate, simple, convenient, minimally invasive, and low-pain.

The interstitial fluid is a type of extracellular fluid that resides between cells and intravascular fluid. It fills the cellular spaces of tissues and serves as one of the crucial fluid environments for maintaining normal cell function. The interstitial fluid contains biomarkers such as glucose and exchanges substances with blood plasma through the tiny pores in the capillary wall. This results in a strong correlation between the concentration of these markers in the interstitial fluid and their concentration in the blood, thus indicating potential value for bioinformatic analysis. Therefore, the indirect detection of blood glucose concentration through the measurement of glucose concentration in interstitial fluid has emerged as a viable solution.

With the rapid advancement of micro-nano processing technology, porous microneedle array has emerged as a novel type of minimally invasive transdermal device, demonstrating unique advantages in the field of biosensing. Particularly, they excel in the extraction of interstitial fluids from the skin, holding significant potential value.

### SUMMARY OF INVENTION

In order to enhance the current conventional blood glucose testing which involves cumbersome operation and causes obvious pain, this application presents a porous microneedle array-based interstitial fluid glucose detection device.

The present application offers a technical solution for a porous microneedle array-based interstitial fluid glucose detection device.

A porous microneedle array-based interstitial fluid glucose detection device includes:
A microneedle module includes a porous microneedle array designed to penetrate the human epidermis and extract interstitial fluid. Its structure includes a microneedle substrate and multiple porous microneedles arranged on the microneedle substrate. The porous microneedles exhibiting a porosity gradient which shows a gradual increase in porosity from the tip of the needle to the microneedle substrate;
A colorimetric detection module includes a colorimetric film designed to produce a color reaction with glucose in the interstitial fluid. The depth of the resulting color is directly correlated to the concentration of glucose;
An electrochemical detection module includes a flexible substrate and a three-electrode system disposed on the flexible substrate. The three-electrode system includes a counter electrode, a reference electrode, and a working electrode. Glucose in the interstitial fluid undergoes a redox reaction at the working electrode, generating a current signal that is correlated with the glucose concentration;
A liquid transport layer, attached to the microneedle substrate, is responsible for transferring interstitial fluid collected by the porous microneedle array to both the colorimetric detection module and the electrochemical detection module.

The porous microneedles in the porous microneedle array exhibit a porosity gradient, with their porosity gradually increasing from the tip to the microneedle substrate. The smaller porosity at the needle tip provides greater strength, resulting in improved puncture ability and reduced pain during insertion. Conversely, the larger porosity along the needle body enables efficient drawing of interstitial fluid, thereby providing adequate samples for colorimetric and electrochemical detection.

The porous microneedle array draws interstitial fluid and then transfers it from the liquid transport layer to both the colorimetric detection module and the electrochemical detection module. In the colorimetric detection module, a colorimetric film reacts with the glucose in the interstitial fluid to produce a color change. The depth of this color change correlates with the concentration of glucose present, enabling rapid and accurate detection of glucose levels. In the electrochemical detection module, glucose in the interstitial fluid undergoes a redox reaction on the working electrode, generating a current signal that is related to the glucose concentration. This enables precise detection of glucose concentration.

The detection device provided in this application is capable of performing both rapid and precise testing of interstitial fluid glucose concentration. The colorimetric and electrochemical testing modules can be used simultaneously or separately for different application scenarios. The colorimetric testing module is suitable for scenarios that require rapid testing, while the electrochemical testing module is more appropriate for scenarios that require precise testing. This provides diabetic patients and those at risk with a convenient solution to accurately assess their blood glucose level.

Furthermore, the porous microneedles are in the shape of a quadrangular pyramid.

The smaller tip of the 4-sided pyramid makes it easier to puncture the skin than normal cones. Additionally, the tip-guiding properties of its shape and structure help to direct the transfer of interstitial fluid from the skin to the microneedle substrate.

Furthermore, the porous microneedles have a micron-scale spatial mesh structure.

The porous structure obtained by the conventional porogenic agent method typically exhibits uneven pore distribution, local instability, and susceptibility to collapse under stress. In contrast, the pores of the porous microneedles provided in this application demonstrate spatial reticulation connectivity, resulting in superior mechanical stability and excellent water-drawing performance.

Furthermore, the method for preparing the porous microneedle array with a porosity gradient involves the following steps:
Male mold preparation: fabricating a male mold of the microneedle array by 3D printing method;
Female mold preparation: cover the female mold material on the surface of the male mold of the microneedle array, and obtain the female mold of the microneedle array after demolding;
Solvent evaporation: inject the PGA-HFIP solution into the female mold of the microneedle array. Subsequently, porous microneedle array with porosity gradient were obtained after the directional evaporation of HFIP.

In addition, during the solvent evaporation step, the solvent at the needle body portion is evaporated first, followed by the evaporation of the solvent at the tip portion.

During the rapid volatilization of the HFIP solvent, the PGA-HFIP solution becomes saturated, particularly at the solid-liquid-gas interface, where localized areas of high saturation develop. Then a small amount of PGA precipitates out to form crystalline nuclei. Subsequently, the PGA component of the solution grows around these crystalline nuclei. As a kind of semi-crystalline material, solid PGA growth without directions, eventually leading to the development of high porosity characterization. As the concentration of PGA increases, the degree of supersaturation of the solution also increases, resulting in more nuclei and a faster rate of PGA precipitation. However, this also leads to a decrease in the volume of the single crystalline region, ultimately forming the final spatial reticulation structure. The final spatial mesh structure formed has a lower porosity.

During the solvent evaporation step in the preparation of porous microneedle array, the solvent near the microneedle substrate evaporates first, followed by the evaporation of the solvent near the tip. This results in a directed manner of solvent evaporation, with slower evaporation and higher residual concentration of PGA-HFIP solution closer to the microneedle tip. Consequently, this leads to precipitation of PGA and lower porosity. By controlling the direction of solvent evaporation in this manner, porous microneedle array with a gradient in porosity can be prepared.

Furthermore, the colorimetric film is a TMB-functionalized porous film that has been immobilized with enzymes of GOx and HRP. Additionally, this colorimetric film is attached to the liquid transport layer.

During the detection process, the interstitial fluid diffuses within the colorimetric film. Then the glucose in the interstitial fluid is oxidized by GOx to produce hydrogen peroxide. Subsequently, under the action of HRP, TMB reacts to generate a colored product. The concentration of glucose directly correlates with the intensity of color produced; a higher concentration results in a darker color. By assessing the depth of color, it is possible to determine the concentration of glucose accurately and effectively.

Furthermore, the method for preparing the colorimetric film involves the following steps:
TMB was dissolved in a PGA-HFIP solution, and then spread onto the substrate. The HFIP was allowed to evaporate, resulting in the formation of a TMB-functionalized porous film;
Colorimetric films were prepared by adding GOx and HRP solutions dropwise to TMB-functionalized porous films.

During the process of solvent evaporation, PGA and TMB were simultaneously precipitated. This allowed for TMB to be uniformly distributed and fixed within the spatial mesh structure of PGA, thereby significantly reducing the “coffee ring effect”. Consequently, this contributes to the enhancement of uniformity and stability in color development for colorimetric detection.

Furthermore, the three-electrode system is coated with a hydrogel layer that is firmly attached to the liquid transport layer.

Furthermore, the working electrode includes a conductive circuit in a cross-combed structure and a porous conductive carrier that covers the conductive circuit. This porous conductive carrier is loaded with GOx.

Furthermore, the porous conductive carrier is a PGA porous particle that has been adsorbed with carbon nanotubes.

The glucose present in the interstitial fluid undergoes a reaction with the GOx located in the working electrode, resulting in the production of hydrogen peroxide. This hydrogen peroxide then undergoes oxidation and decomposition to generate electrons when a specific potential is externally applied. The higher the concentration of glucose, the greater the production of hydrogen peroxide. In other words, as more electrons are generated, a higher current is produced. By detecting the magnitude of the current, it is possible to analyze the concentration of glucose in the interstitial fluid.

The cross-comb structure of the conductive circuit enhances the contact area between the solution to be measured and the working electrode. The porous conductive carrier possesses a three-dimensional structure with high surface area and excellent biocompatibility, enabling tight immobilization of the enzyme GOx while maintaining its activity. This design increases the contact area of the reaction, resulting in a larger detection current under equivalent substrate concentrations. Consequently, it improves signal resolution and detection accuracy, facilitating highly sensitive detection of glucose oxidation enzyme in mesophilic fluid, as well as highly sensitive detection of glucose in interstitial fluid.

In conclusion, the current application encompasses at least one of the following advantageous technical effects:
1. The porous microneedles in the porous microneedle array provided by the present application feature a micrometer-scale spatial mesh structure and a porosity gradient. The porosity gradually increases from the tip of the needle to the microneedle substrate, with the tip having a smaller porosity for greater strength and puncture ability. Meanwhile, the needle body with larger porosity exhibits good water-drawing ability, enabling rapid extraction of mesenchymal fluid to provide ample material for colorimetric and electrochemical testing. This design is conducive to improving detection efficiency;
2. The colorimetric detection module provided in this application utilizes the principle of color response change to determine the concentration of a specific substance. It does so by analyzing the degree of color change caused by the substance, which is highly selective and sensitive. This module is capable of making a rapid response to changes in the concentration of the target substance and carrying out accurate measurement, facilitating the user's rapid access to the detection results;
3. The electrochemical detection module circuit provided in this application is manufactured using inkjet printing technology, a simple and easily scalable process. The porous conductive carrier in the working electrode features a three-dimensional structure with high specific surface area and excellent biocompatibility. This structure effectively immobilizes the GOx enzyme while maintaining its activity, thereby increasing the contact area for reactions. As a result, larger detection currents are obtained at the same concentration of the target substance, leading to improved signal resolution and detection accuracy. This enables high sensitivity detection of glucose in interstitial fluid;
4. The porous microneedle array-based interstitial fluid glucose detection device provided in this application, features a simple structure and easy operation. It is capable of minimally invasive and low-pain puncture of the skin, enabling real-time detection on-portion. The device allows for both rapid and accurate detection of interstitial fluid glucose concentration. Additionally, the colorimetric detection module and electrochemical detection module can be used either simultaneously or separately. This provides diabetic patients and individuals at risk with a convenient solution for accurately assessing their blood glucose levels. Furthermore, it can be applied to practical scenarios such as self-testing of blood glucose concentration and rapid blood glucose detection in diabetic patients.

### BRIEF DESCRIPTION OF DRAWINGS

FIG. 1 depicts a side view of a porous microneedle array-based interstitial fluid glucose detection device in an embodiment of the present application;

FIG. 2 depicts a top view of a porous microneedle array-based interstitial fluid glucose detection device in an embodiment of the present application;

FIGS. 3A-3C depict partial views of a porous microneedle with a porosity gradient in an embodiment of the present application. FIG. 3A represents the tip portion, FIG. 3B denotes the middle part of the needle body, and FIG. 3C indicates a portion near the microneedle substrate;

FIG. 4 depicts the results of the colorimetric assay in this particular embodiment of the present application, evaluating the performance of simulated interstitial fluid with a concentration range of 0-10 mM glucose;

FIG. 5 depicts the results of electrochemical testing at 0-10 mM glucose for simulated interstitial fluid performance in embodiments of the present application.

### DESCRIPTION OF EMBODIMENTS

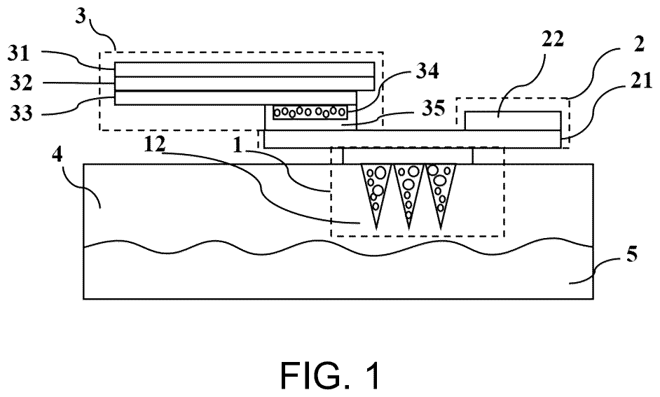

The present application will be further detailed below in conjunction with the accompanying FIGS. 1-5.

The present application discloses embodiments of a porous microneedle array-based interstitial fluid glucose detection device. Referring to FIG. 1, the porous microneedle array-based interstitial fluid glucose detection device includes a microneedle module 1, a colorimetric detection module 2, an electrochemical detection module 3, and a liquid transport layer 21.

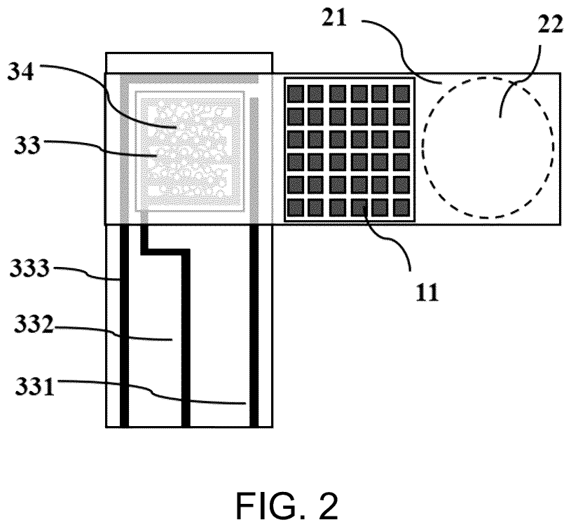

Referring to FIGS. 1 and 2, the microneedle module 1 includes a porous microneedle array 11 designed for piercing the human epidermis and extracting interstitial fluid. The porous microneedle array 11 includes a microneedle substrate and multiple porous microneedles 12 arranged on the microneedle substrate, with the porous microneedles 12 exhibiting a porosity gradient. The porosity increases along the direction from the needle tip towards the microneedle substrate.

The method of preparing a porous microneedle array 11 with the porosity gradient involves the following steps.

Step 1, male mold preparation: prepare a male mold of the microneedle array through 3D printing. The substrate size is 10 mm×10 mm, with a total of 36 quadrangular pyramid-shaped microneedles uniformly distributed in a 6×6 pattern. The spacing between adjacent microneedles is 1 mm. Each microneedle substrate has a length and width of 480 μm, a height of 900 μm, and a tip angle of 30°.

Step 2, female mold preparation: cover the female mold material, Polydimethylsiloxane (PDMS), on the surface of the male mold of the microneedle array. The female mold of the microneedle array was then obtained after demolding.

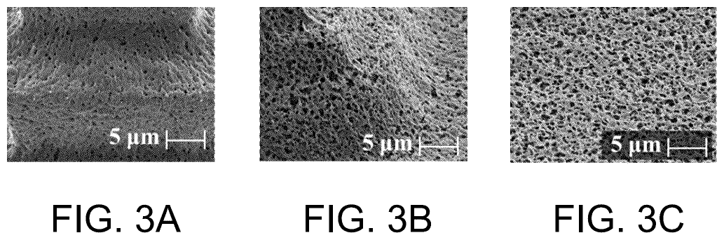

Step 3, solvent evaporation: inject PGA-HFIP solution (with a concentration of 50 mg/mL) into the female mold of the microneedle array. The female mold of the microneedle array was then placed upright, with the open end facing upward and the needle tips pointing downward. The HFIP solvent evaporates in a directional manner, the solvent in the needle body evaporating first, and the solvent in the needle tip portion evaporates later. This process resulted in porous microneedle array with spatial reticulation at a micrometer level and a porosity gradient. As depicted in FIGS. 3A-3C, the porosity increased gradually along the direction from the tip to the microneedle substrate, with a porosity gradient ranging from 7% to 48%.

The tip of the needle with smaller porosity exhibits greater strength, resulting in enhanced puncture ability and reduced pain during the procedure. On the other hand, the needle body with larger porosity demonstrates excellent water-drawing capability, enabling rapid extraction of mesenchymal fluid to provide a sufficient sample for colorimetric and electrochemical detection. This facilitates efficient and accurate testing processes.

Referring to FIGS. 1 and 2, the liquid transport layer 21 is a PGA porous membrane attached to the microneedle substrate. It has an average thickness of 49 μm, with distributed pores ranging from 1-5 μm in diameter and a porosity of 42%. The liquid transport layer 21 serves the purpose of transferring the interstitial fluid drawn by the porous microneedle array 11 to both the colorimetric detection module 2 and the electrochemical detection module 3 simultaneously.

Referring to FIGS. 1 and 2, the colorimetric detection module 2 includes a colorimetric film 22 designed for color development in reaction with glucose in the interstitial fluid. The colorimetric film 22 is a TMB-functionalized porous film that has been fixed with GOx and HRP, and it is adhered to the liquid transport layer 21.

The method of preparing a colorimetric film involves the following steps:

Step 1: dissolve 10 mg of TMB in 2 mL of PGA-HFIP solution, with a PGA concentration of 50 mg/mL. Mix well and spread the solution on the substrate to allow for evaporation of HFIP, resulting in a TMB-functionalized porous film and a porosity of 43%;
During the process of solvent evaporation, PGA and TMB are precipitated simultaneously. This allows TMB to be evenly distributed and fixed within the spatial mesh structure of PGA, effectively reducing the “coffee ring effect”. As a result, this contributes to enhancing the uniformity and stability of color development in colorimetric films.

Step 2: A total of 2 μL of a solution containing GOx and HRP (both enzymes at a concentration of 100 U/mL) was carefully added drop by drop onto the TMB-functionalized porous film. This allowed for the adsorption of GOx and HRP into the porous structure of the TMB-functionalized film, resulting in the formation of a colorimetric film.

Referring to FIGS. 1 and 2, the electrochemical detection module 3 includes a flexible substrate and a three-electrode system positioned on the flexible substrate. The three-electrode system includes a counter electrode 333, a reference electrode 331, and a working electrode 332. This three-electrode system is enclosed within a hydrogel 35, which is attached to the liquid transport layer 21.

Referring to FIGS. 1 and 2, the flexible substrate comprises a lower substrate 31 and an upper substrate 32. The lower substrate 31 is a Polyimide (PI) film with a thickness of 50 μm and dimensions of 15 mm×5 mm, while the upper substrate 32 is a PDMS film with a thickness of 70 μm and dimensions of 15 mm×5 mm. The upper substrate 32 undergoes inkjet printing with nano-silver conductive circuits 33, featuring a line width of 100 μm after oxygen plasma modification treatment. The flexible substrate, printed with nano-silver conductive circuits 33, is then sintered and cured at 150° C. for the preparation of the reference electrode 331, the counter electrode 333, and the working electrode 332:

The reference electrode 331 and counter electrode 333 are Ag/AgCl electrodes obtained through the chlorination of nanosilver conductive circuits 33.

Referring to FIGS. 1 and 2, the working electrode 332 includes a conductive circuit arranged in a cross-combed structure with a tooth spacing of 150 μm and an average resistivity of 2.7 Ω/mm. The cross-combed structure of the conductive circuit is coated with a porous conductive carrier 34 that has been fixed with enzymes. The porous conductive carrier is PGA porous particles adsorbed with carbon nanotubes loaded with GOx.

The preparation method for the PGA porous particles is as follows: slowly add an equal amount of water to a 50 mg/mL PGA-HFIP solution while stirring gently, generating PGA flocculent material; then high-speed stir to break up the PGA flocculent material, obtaining PGA porous particles. By controlling the duration of high-speed stirring, PGA porous particles of different particle size ranges may be obtained. In the embodiment, the high-speed stirring time is 48 h, resulting in PGA porous particles with a particle size of 10-20 μm.

Carbon nanotubes were mixed with PGA porous particles, and then GOx was added to obtain a porous conductive carrier 34 with fixed enzyme. This carrier was coated on the conductive circuits of the cross-comb structure in the region of the working electrode 332 to complete the preparation of the working electrode 332. Finally, the three-electrode system was encapsulated using hydrogel 35 to complete the preparation of electrochemical detection module 3.

In the aforementioned steps of preparing porous microneedle array, colorimetric films, and porous particles of PGA, the PGA-HFIP solution was prepared as follows:

The PGA particles were encased in aluminum foil and then placed on the chassis of a hot press with a preheating temperature of 230° C. They were heated for 100 seconds to completely melt the PGA particles. Subsequently, the temperature was maintained and the hot press was operated to apply pressure for another 100 seconds at a set pressure. After the hot press treatment, the aluminum foil wrapped with PGA was removed from the hot press and allowed to cool for 15 seconds at room temperature. A low-crystallinity PGA film was then peeled off from the aluminum foil. The low crystallinity PGA film was dissolved in HFIP to obtain a PGA-HFIP solution.

The PGA particles, with a diameter of a few millimeters, are pressed into a film hundreds of microns thick through hot pressing. This process dramatically increases the heat exchange area and enables rapid cooling in room temperature air. Simultaneously, the high thermal conductivity characteristics of the aluminum foil are utilized to quench and cool the PGA film in the air. This effectively blocks the recrystallization process of the PGA, thereby reducing its crystallinity. As a result, the PGA can easily be dissolved in organic solvents such as HFIP.

After the completion of the preparation of the microneedle module 1, the colorimetric detection module 2 and the electrochemical detection module 3, these modules are assembled into an interstitial fluid glucose detection device and attached to the human skin. The porous microneedle 12 pierces the epidermal layer 4 of the skin, allowing interstitial fluid to migrate to the microneedle substrate through the porous channels within the porous microneedle array 11. Subsequently, it is rapidly delivered by the liquid transport layer 21 to the colorimetric detection module 2 and the electrochemical detection module 3.

In the colorimetric detection module 2, the interstitial fluid diffuses into the colorimetric film 22. The GOx then catalyzes the oxidation of glucose in the interstitial fluid to produce hydrogen peroxide. Subsequently, the TMB undergoes a reaction under the influence of HRP, resulting in the production of a colored product. It is worth noting that as the concentration of glucose increases, more colored products are generated, leading to a darker coloration. Therefore, by assessing the depth of color, it is possible to determine and quantify the concentration of glucose.

In the electrochemical detection module 3, glucose in the interstitial fluid undergoes a reaction with GOx in the working electrode to produce hydrogen peroxide. This hydrogen peroxide then oxidizes and decomposes to generate electrons when a certain potential is externally applied. The concentration of glucose directly correlates with the amount of hydrogen peroxide produced, leading to an increase in electron generation and subsequently, current generation. By analyzing the magnitude of the current, it is possible to detect and obtain the concentration of glucose in the interstitial fluid.

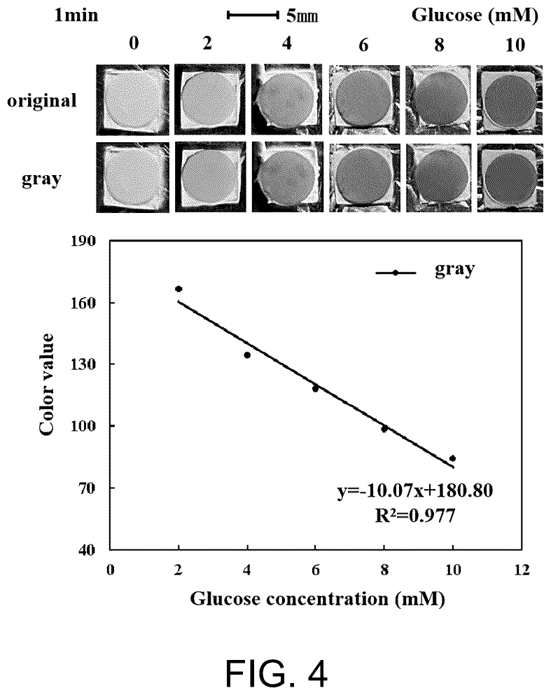

The results of the colorimetric detection module 2 on the simulated interstitial fluid containing 0-10 mM glucose are presented in FIG. 4. The detection exhibited a uniform color distribution, with no significant areas of color variation, and a clear color gradient corresponding to different glucose concentrations. The calibration curve of the gray scale value of the developed color against the glucose concentration demonstrated excellent linearity, encompassing the linear detection range of typical fluctuations in human blood glucose levels, with a sensitivity of 10.07/mM and R²=0.977. The specific details regarding detection accuracy can be found in Table 1, which indicates a detection accuracy of 96.2%.

TABLE 1
Detection accuracy results of the colorimetric detection module

| Gray scale values | | | | | | Average |
|---|---|---|---|---|---|---|
| and accuracy | 2 mM | 4 mM | 6 mM | 8 mM | 10 mM | value |
| Calibration experiment | 174.73 | 131.72 | 119.04 | 105.24 | 80.06 | — |
| grayscale value | | | | | | |
| Detection of the | 166.69 | 134.58 | 118.14 | 98.33 | 84.09 | — |
| application gray value | | | | | | |
| Detection accuracy | 95.4% | 97.8% | 99.2% | 93.4% | 95.0% | 96.2% |

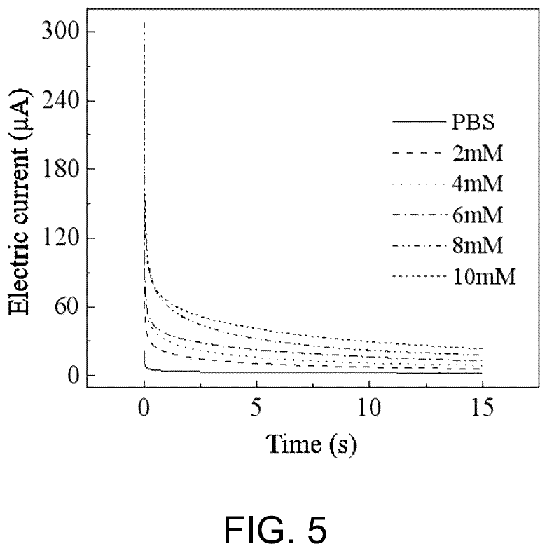

The electrochemical detection module 3 demonstrates its detection effect on simulated interstitial fluid containing 0-10 mM glucose in FIG. 5. The response current increases with the rise in glucose concentration, and significant differentiation is observed in the response current at s. Table 2 presents the specific values of detection accuracy, indicating an average detection accuracy of 97.8% for the electrochemical detection module.

TABLE 2
Detection accuracy results of the electrochemical detection module

| | Detection | Test | Actual | Detection |
|---|---|---|---|---|
| Experiment | current | concentration | concentration | accuracy |
| number | (μA) | (mM) | (mM) | (%) |
| 1 | 1.891 | 0.046 | 0 | — |
| 2 | 6.954 | 1.971 | 2 | 98.6 |
| 3 | 12.024 | 3.899 | 4 | 97.5 |
| 4 | 17.353 | 5.925 | 6 | 98.8 |
| 5 | 23.307 | 8.189 | 8 | 97.6 |
| 6 | 29.043 | 10.370 | 10 | 96.3 |
| Average value | — | — | — | 97.8 |

The current application is capable of rapidly and accurately detecting the concentration of interstitial fluid glucose, making it suitable for various application scenarios. It offers a convenient and precise solution for diabetic patients and individuals at risk to assess blood glucose levels with ease and accuracy.

All of the aforementioned examples represent superior embodiments of the current application and are not intended to restrict the scope of protection. Therefore, any modifications that are equivalent and made in accordance with the structure, shape, and principles of the present application shall fall within the protective scope thereof.

What is claimed is:

1. A porous microneedle array-based interstitial fluid glucose detection device, comprising:
a microneedle module comprising a porous microneedle array configured to penetrate human epidermis and extract interstitial fluid, wherein the porous microneedle array includes a microneedle substrate and a plurality of porous microneedles arranged on the microneedle substrate, the porous microneedles have a porosity gradient, and the porosity gradient gradually increases from a needle tip portion towards the microneedle substrate;
a colorimetric detection module comprising a colorimetric film configured to react with glucose in the interstitial fluid to develop color, a depth of the color is directly correlated to a glucose concentration;
an electrochemical detection module comprising a flexible substrate and a three-electrode system disposed on the flexible substrate, wherein the three-electrode system comprises a counter electrode, a reference electrode, and a working electrode, glucose in the interstitial fluid undergoes a redox reaction at the working electrode, generates a current signal that is directly correlated with the glucose concentration; and
a liquid transport layer, attached to the microneedle substrate, configured for transferring the interstitial fluid collected by the porous microneedle array to both the colorimetric detection module and the electrochemical detection module.

2. The porous microneedle array-based interstitial fluid glucose detection device according to claim 1, wherein each of the porous microneedles is in the shape of a quadrangular pyramid.

3. The porous microneedle array-based interstitial fluid glucose detection device according to claim 1, wherein each of the porous microneedles has a micron-scale spatial mesh structure.

4. A method of preparing porous microneedle array with porosity gradient, comprising:
male mold preparation: preparing a male mold of a microneedle array through 3D printing;
female mold preparation: covering a female mold material on a surface of the male mold of the microneedle array, and obtaining a female mold of the microneedle array after demolding;
solvent evaporation: injecting a Polyglycolide acid (PGA)-Hexafluoroisopropanol (HFIP) solution into the female mold of the microneedle array, and obtaining a porous microneedle array with a porosity gradient after directional evaporation of HFIP.

5. The method of preparing porous microneedle array with porosity gradient according to claim 4, wherein in a step of the solvent evaporation, a solvent in a needle body portion evaporates first, and the solvent in the needle tip portion evaporates later.

6. The porous microneedle array-based interstitial fluid glucose detection device according to claim 1, wherein: the colorimetric film is a Tetramethylbenzidine (TMB)-functionalized porous film which Glucose oxidase (GOx) and Horseradish peroxidase (HRP) are fixed on, and this colorimetric film is attached to the fluid conducting layer.

7. A method of preparing colorimetric film, comprising:
dissolving TMB in a PGA-HFIP solution and spreading it on a substrate, evaporating HFIP to obtain a TMB functionalized porous film; and
adding glucose oxidase and horseradish peroxidase solution dropwise onto the TMB functionalized porous film to obtain a colorimetric film.

8. The porous microneedle array-based interstitial fluid glucose detection device according to claim 1, wherein the three-electrode system is covered with a hydrogel layer, and the hydrogel layer is attached to the liquid transport layer.

9. The porous microneedle array-based interstitial fluid glucose detection device according to claim 1, wherein the working electrode comprises a conductive circuit in a cross-combed structure and a porous conductive carrier covering the conductive circuit, and the porous conductive carrier is loaded with GOx.

10. The porous microneedle array-based interstitial fluid glucose detection device according to claim 9, wherein the porous conductive carrier comprises PGA porous particles adsorbed with carbon nanotubes.
